(19) United States
(12) Patent Application Publication
(10) Pub. No.: US 2015/0196612 A1
(43) Pub. Date: Jul. 16, 2015

(54) Anti-enterovirus 71 steroidal saponins and their preparation

(71) Applicants: Shanghai University of Traditional Chinese Medicine, Shanghai (CN); Hong Kong Baptist University, Hong Kong (HK)

(72) Inventors: Quanbin HAN, Hong Kong (HK); Aiping LU, Hong Kong (HK); Zhaoxiang BIAN, Hong Kong (HK); Hongxi XU, Hong Kong (HK); Mengshun LIU, Hong Kong (HK); Ling TAO, Hong Kong (HK); Mengjie WANG, Hong Kong (HK); Kaixian CHEN, Hong Kong (HK)

(73) Assignees: Shanghai University of Traditional Chinese Medicine, Shanghai (CN); Hong Kong Baptist University, Hong Kong (HK)

(21) Appl. No.: 14/151,832

(22) Filed: Jan. 10, 2014

(51) Int. Cl. A61K 36/88

(52) U.S. Cl. CPC A61K 36/88

(57) ABSTRACT

The present invention discloses a novel composition with anti-viral effects comprises extracts from Anemarrhena asphodeloides. Method of preparing said extract is also disclosed.

### FIELD OF INVENTION

The present invention relates to a type of novel steroidal saponins and a method to prepare such steroidal saponins by extraction of Anemarrhena asphodeloides, and their anti-enterovirus 71 activity.

### BACKGROUND OF INVENTION

Hand, foot and mouth disease (Hand, foot and mouth disease, HFMD) is an infectious disease caused by enterovirus, mostly occurs in infants and young children with a localized rash, but some patients develop infection of the central nervous system, even to death. HFMD infected 1,520,274 people with 431 deaths reported up to end of July in 2012 in China. Although a significant degree of uncertainty exists with reference to the diagnosis, World Health Organization states “Based on the latest laboratory results, a significant proportion of the samples tested positive for enterovirus 71 (EV-71), which causes hand foot and mouth disease (HFMD). The EV-71 virus has been known to generally cause severe complications amongst some patients.”(WHO: http://www.wpro.who.int/emerging_diseases/HFMD/en/index.html; retrieved on 2 Oct., 2013)

EV71 outbreaks have been associated with a variety of severe neurological complications that can deteriorate rapidly to involve cardiopulmonary failure with high mortality rates. There is currently no effective vaccine or antiviral against EV71. Treatments for acute EV71 infections with neurological manifestations mainly aim to alleviate symptoms. Ribavirin has been used currently for treatment of EV71 infection. However, ribavirin could not increase the survival rates of infected subjects. Therefore, there is an urgent need to develop effective treatment to prevent or reduce EV71-related deaths.

Previous studies showed in Bae, G; Lee, J. R.; Chang, J.; Seo, E. K.; Identification of nyasol and structurally related compounds as the active principles from Anemarrhena asphodeloides against respiratory syncytial virus (RSV). Chem. Biodivers. 2007, 4, 2231-5 that the components of Anemarrhena asphodeloides a well-known traditional Chinese medicinal herb, exerted various antiviral activities. Timosaponin A-III showed in Youn, U. J. & Jang J. E.; Anti-respiratory syncytial virus (RSV) activity of timosaponin A-III from the rhizomes of Anemarrhena asphodeloides. J. Med. Plants. Res. 2011; 5: 1062-5 that potent inhibitory effects on the respiratory syncytial virus (RSV), with an IC50 value of 1.00 μM. A further investigation of Mangiferin shown in Wang, R. R.; Gao, Y. D.; Ma, C. H.; Mangiferin, an Anti-HIV-1 Agent Targeting Protease and Effective against Resistant Strains. Molecules 2011, 16, 4264-4277 that could inhibit Human immunodeficiency virus I (HIV-1) induced syncytium formation at non-cytotoxic concentrations, with a 50% effective concentration (EC50) at 16.90 μM and a therapeutic index (TI) above 140 (Wang R R & Gao Y D Molecules 2011;16: 4264-4277). However, knowledge about anti-EV71 compounds from Anemarrhena asphodeloides is quite limited. Our invention disclosed in this anti-EV71 compound is novel and not known in the art.

Citation or identification of any reference in this section or any other section of this application shall not be construed as an admission that such reference is available as prior art for the present application.

### SUMMARY OF INVENTION

Accordingly, it is an object of the present invention to provide a composition with anti-viral effect comprising steroidal saponins extracted from herbal plants. In the first aspect of the present invention there is provided the steroidal saponins comprising mangiferin, timosaponin B-II, anemarsaponin B, anemarsaponin II, timosaponin G, timosaponin A-IV and timosaponin A-III.

In the second aspect of the present invention there is provided the herbal plants comprising Anemarrhena asphodeloides.

In the third aspect of the present invention there is provided the anti-viral effects comprising anti-enteroviral effects. In one embodiment, the anti-enteroviral effect is anti-enterovirus 71 effect.

In the fourth aspect of the present invention there is provided the extraction method comprising the use of bioassay-guided counter-current chromatography.

In one embodiment of the fourth aspect of the present invention there is provided the counter-current chromatography is performed more than once.

In another embodiment of the fourth aspect of the present invention there is provided the counter-current chromatography extraction method comprising at least one approach based on the mechanism of continued solvent partition.

In yet another embodiment of the fourth aspect of the present invention there is provided the solvent partition comprising at least one two-phase solvent system which comprising ethyl acetate/butanol/water.

In the fifth aspect of the present invention there is provided a use of the composition of the present invention in the treatment of virus induced diseases.

In one embodiment of the fifth aspect of the present invention there is provided the virus induced diseases comprising diseases caused by enterovirus 71, such as hand, foot and mouth disease.

Those skilled in the art will appreciate that the invention described herein is susceptible to variations and modifications other than those specifically described.

The invention includes all such variation and modifications. The invention also includes all of the steps and features referred to or indicated in the specification, individually or collectively, and any and all combinations or any two or more of the steps or features.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

Other definitions for selected terms used herein may be found within the detailed description of the invention and apply throughout. Unless otherwise defined, all other technical terms used herein have the same meaning as commonly understood to one of ordinary skill in the art to which the invention belongs.

Other aspects and advantages of the invention will be apparent to those skilled in the art from a review of the ensuing description.

### BRIEF DESCRIPTION OF DRAWINGS

The present invention will become apparent from the following description, when taken in conjunction with the accompanying drawings, in which:

FIG. 1 shows the chemical structures of isolated compounds 1-7 from Anemarrhena asphodeloides and reference compounds 8-14 of spirostanol sapogenins or saponins.

FIG. 2 shows the fractionation of crude extract of Anemarrhena asphodeloides by a first bioassay-guided counter-current chromatography (CCC1) into fractions CCC1 I-V (FIG. 2A) and the anti-EV71 activity of fractions CCC1 I-V, crude extract and ribavirin (FIG. 2B).

FIG. 3 shows the fractionation of fraction CCC1 V by a second counter-current chromatography (CCC2) into fractions CCC2 I-XII and the anti-EV71 activity of fractions CCC2 I-XII (FIG. 3A). Fraction CCC2 XII is fractionated by a third counter-current chromatography (CCC3) into fractions CCC3 I-V and the anti-EV71 activity of fractions CCC3 I-V (FIG. 3B)

FIG. 4 shows the fractionation of fraction CCC1 IV by a forth counter-current chromatography (CCC4) into fractions CCC4 1-51 and the anti-EV71 activity of fractions CCC4 1-51.

FIG. 5 shows NMR spectral analysis of Compounds 2-7 of the present invention.

FIG. 6 shows cytotoxicity and anti-viral activity of compounds 2-7 of the present invention, reference compounds 8-14 of spirostanol sapogenins and Ribavirin (positive control)

### DETAILED DESCRIPTION OF INVENTION

The present invention is not to be limited in scope by any of the specific embodiments described herein. The following embodiments are presented for exemplification only.

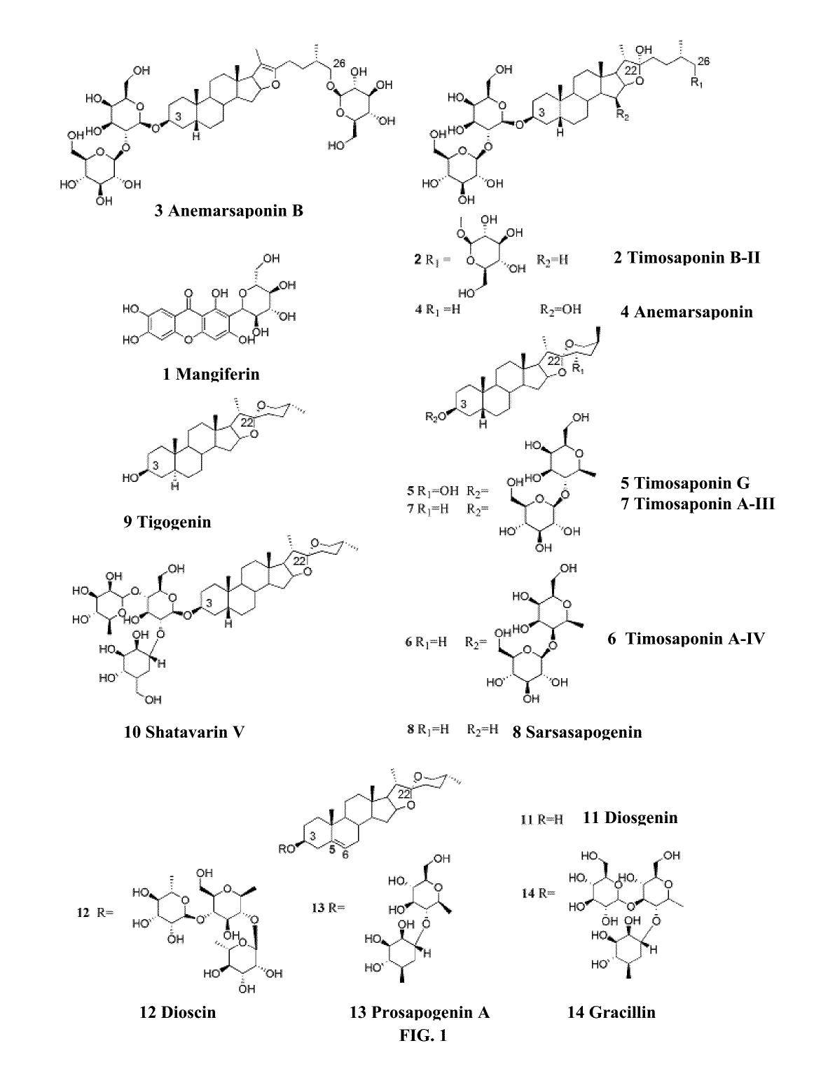

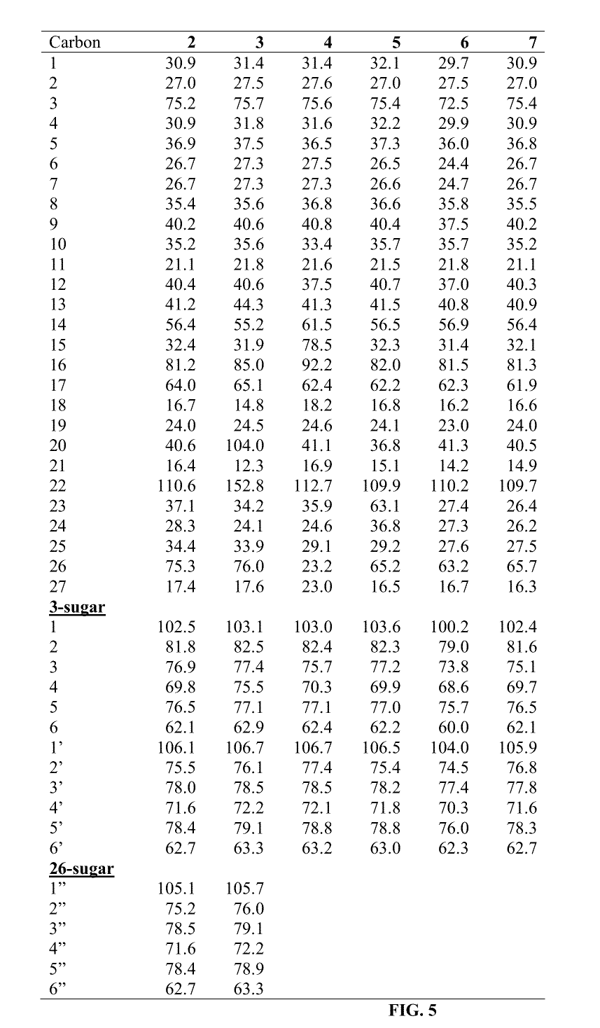

The present invention discloses a composition with anti-viral effects comprises one or more steroidal saponins extracted from Anemarrhena asphodeloides. In one embodiment, the present composition has anti-entroviral effects. The steroidal saponins of the present invention are extracted from crude extraction of Anemarrhena asphodeloides by bioassay-guided counter-current chromatography (CCC). In one embodiment, said one or more steroidal saponins of A. asphodeloides comprises at least one of the following compounds 1-7: mangiferin (compound 1), timosaponin B-II (compound 2), anemarsaponin B (compound 3), anemarsaponin II (compound 4), timosaponin G (compound 5), timosaponin A-IV (compound 6), and timosaponin A-III (compound 7). Chemical structures of compounds 1-7 and NMR spectral analysis of compounds 2-7 of the present invention are shown in FIG. 1 and FIG. 5. In another embodiment, the present anti-viral composition comprises timosaponin BII (compound 2) which exhibits a low IC50 which is only 1% of that of positive control ribavirin and its selectivity index is more than 40 times of ribavirin's.

Current extraction methods like column chromatography are doubtful because irreversible absorptive loss of samples onto the solid separation materials would cause poor sample recovery and high risk in losing active compounds. The present invention discloses extraction of plant and medicinal herbs (in particular A. asphodeloides) using counter-current chromatography (CCC) based on the mechanism of continued solvent partition, guarantee a much higher (>90%) sample recovery rate and bioactivity evaluation of all compounds without missing.

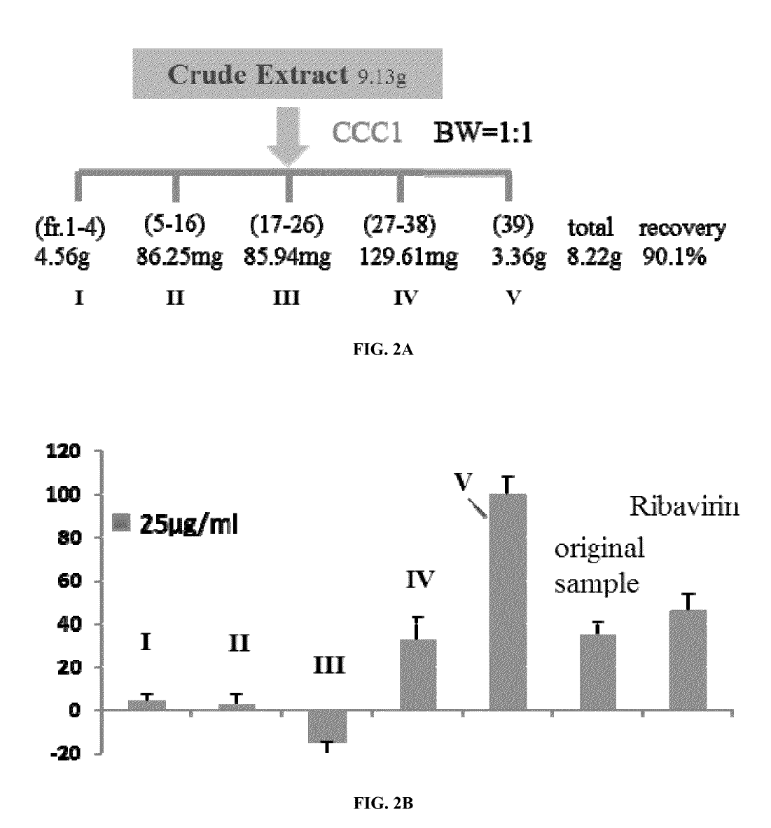

Four CCC fractionations are performed to crude extract of A. asphodeloides. As shown in FIG. 2A, the first CCC fractionation (CCC1) with butanol/water (1:1) affords thirty-nine fractions from the crude extract which are combined into five fractions (fractions no. CCC1 I-V) according to their TLC pattern. Anti-EV71 activities of fractions no. CCC1 I-V are measured and compared with the original crude extract at the concentration of 25 μg/ml and the positive control ribavirin (65 μg/ml) (FIG. 2B). Fraction no. CCC1 V (the part remained in the CCC column) exhibits the strongest anti-viral effect while fraction no. CCC1 IV is comparable with crude extract and positive control. Fractions no. CCC1 I-III (>4.7 g), mainly aqueous chemicals, are shown to be inactive.

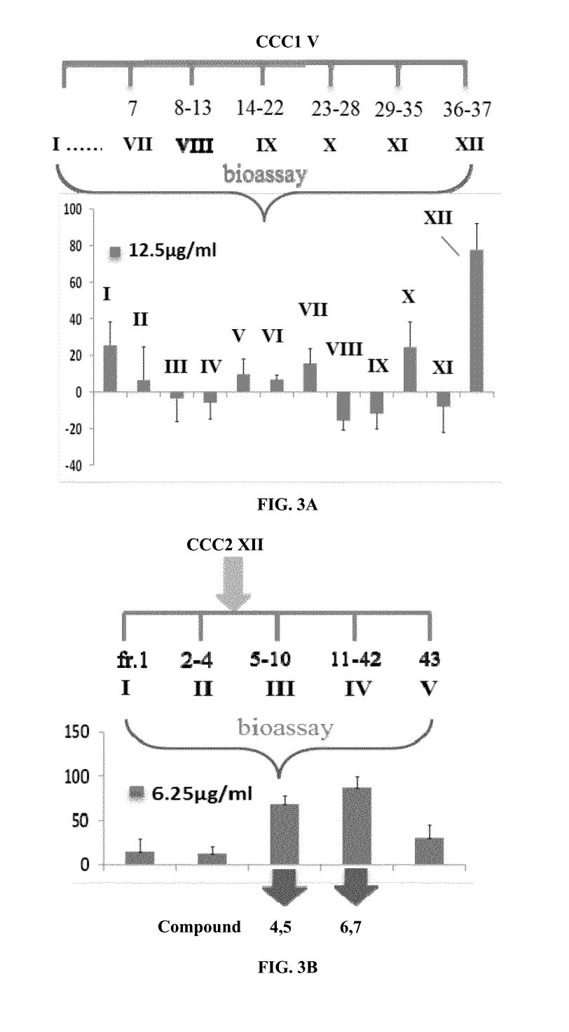

Fraction no. CCC1 V (yielded; 3.08/3.35 g; 92.0% recovery) is further fractionated in second fractionation CCC2 under Ethyl acetate/butanol/water (EBW, 4:1:5) as the two-phase solvent system. Thirty seven fractions are obtained and combined into twelve fractions (Fractions no. CCC2 I-XII) based on TLC examination. Anti-EV71 activities of fractions no. CCC2 I-XII are measured (FIG. 3A). Bioassay at a lower concentration of 12.5 μg/ml of fractions no. CCC2 I-XII show that fraction no. CCC2 XII (the part remained in the CCC 2 column) is the most effective. Fraction no. CCC2 XII fails to directly enter further separation due to insufficient sample amount of only 0.5 g. Fraction no. CCC2 XII is enriched as follows: 15 g of ethanol extract is partitioned with 1 L of ethyl acetate/n-butanol/water (4:1:5) and the upper phase is collected and dried to get 2.31 g of target fraction.

Subsequent third fractionation CCC3 of the enriched fraction no. CCC2 XII (yield: 2.21/2.31 g; 95.8% recovery) yields forty-three fractions using the diphase solvent system of ethyl acetate/methanol/water (5:1:5) and combined into five fractions (Fractions no. CCC3 I-V) for anti-viral bioassay at a further decreased concentration of 6.25 μg/m1 (FIG. 3B). Fractions no. CCC3 III and IV show significant higher antiviral activity than others. Further purification of fractions no. CCC3 III and IV on sephadex column afford compounds 4-7. Compounds 4-6 are identified by their 13C NMR spectra (FIG. 5) to be anemarsaponin II, timosaponin G, timosaponin A-IV, and compound 7 is identified as timosaponin A-III by comparison HPLC chromatography with reference compound using HPLC.

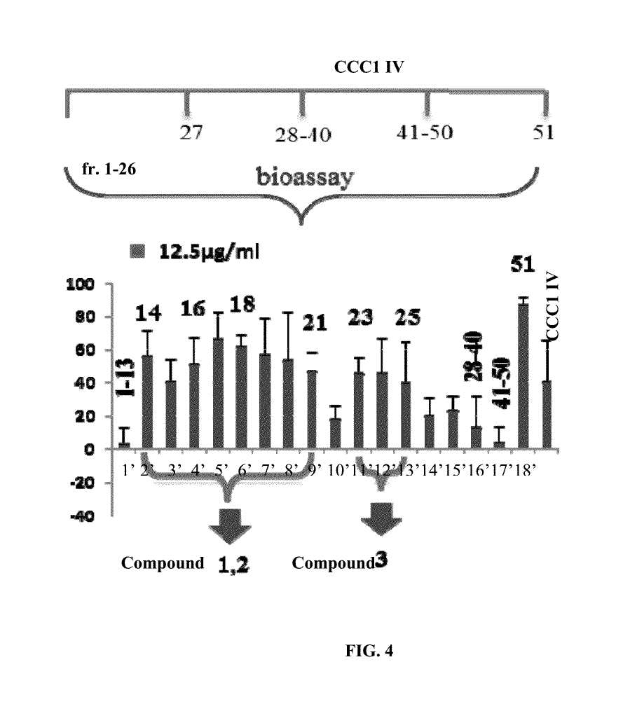

Fraction no. CCC1 IV (yield: 3.96/4.11g; 96.2% recovery) is enriched and is separated by a forth counter-current chromatography CCC4 using the modified ethyl acetate/n-butanol/water (1:4:5) and fifty-one fractions are obtained. The fifty one fractions are further combined into eighteen fractions (fractions no. CCC4 1′-18′), out of which twelve fractions (fraction No. CCC4 2′-9′, 11′-13′, and 18′) are found to be more active than CCC1 IV at the concentration of 12.5 μg/ml (FIG. 4). Purification of the fractions no. CCC4 2′-9′ and 11′-13′ on sephadex column afford compounds 1-3. The fraction no. CCC4 18′ is found to be identical to the already studied CCC2 XII. Compounds 1-3 are identified to be mangiferin, timosaponin B-II, anemarsaponin B based on HPLC comparison with reference chemicals and NMR data (FIG. 5)

The Cytotoxicities of Steroidal Saponins and Related Anti-EV71 Effects

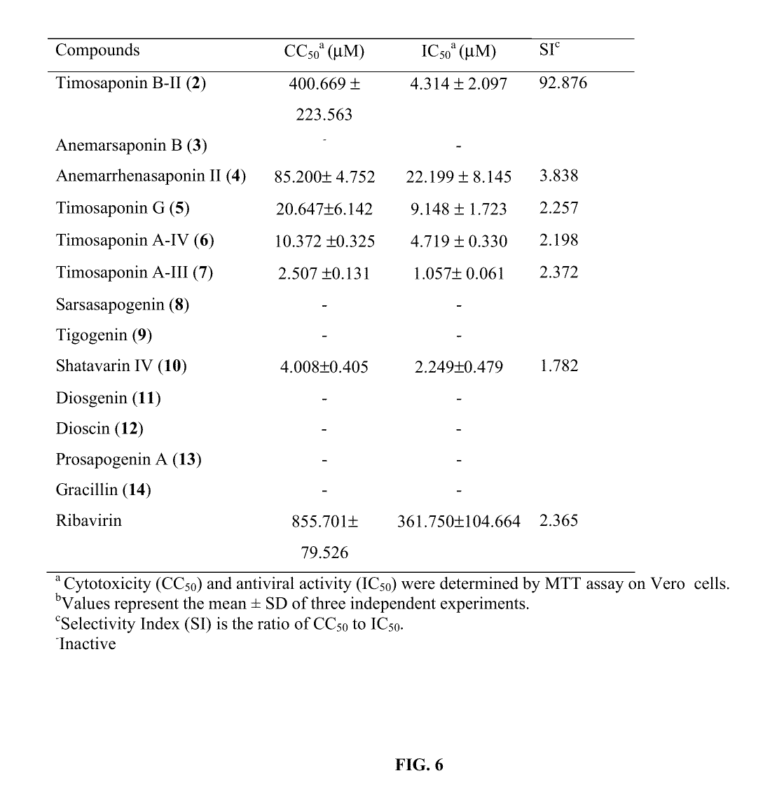

IC50 (50% inhibitory concentration), CC50 50% cytotoxic concentration), and Selective Index (SI) of compound 2-7 are compared with reference compounds 8-14 and the positive control ribavirin. As shown in FIG. 6, compound 3 is completely inactive. The CC50 values of compounds 5-7 are all below 25 μM, whereas compounds 2 and 4 have much higher CC50 values of 400.669 and 85.200 μM, respectively. Ribavirin showed a doubled CC50 compared to compound 2, but its IC50 was also the highest, therefore its SI was only 2.365. Among the isolated saponins, compound 7 shows the lowest IC50 of 1.057 μM, while compound 2 shows a promising SI up to 92.876.

Considering all the active saponins are spirostanol-relevant steroids, it is evidenced that natural products (such as plants and medicinal herbs) with spirostanol skeleton have antiviral activity, and in particular anti-EV71 bioactivity. Some steroid analogues (8-14) are collected together with the isolated saponins to study the structure-activity relationship (FIG. 1). Structure-activity difference between compounds 2 and 3 suggested that dehydration at C22 completely destroyed the activity of furostanol saponin. By contrast, the substitution of alpha-hydroxyl at C23 might decrease both the antiviral activity and the cytotoxicity, while keeping a similar SI value, as indicated by the comparison between compounds 5 and 7. In addition, the part of saccharide also plays important role in the anti-EV71 activities. Removal of the glucose at C26 will significantly decrease the antiviral activity and increase the cytotoxicity as observed in the structure-activity difference between compounds 2 and 4. Based on comparison between 6 and 7, it is further suggested that the change of sugar chain at C3 from 3-O-β-D-glucopyranosyl-(1→2)-β-D-galactopyranosyl in 7 to 3-O-β-D-glucopyranosyl-(1→2)-β-D-mannopyranosyl in 6 significantly decreases both the antiviral activity and the cytotoxicity, while keeping a similar SI value. Sarsasapogenin (8), the aglycon of 6 and 7, is found completely inactive, suggesting that the substitution of disaccharide residue at C3 endows the aglycon with anti-EV71 activities. The contribution of sugar chain is confirmed when another pair of aglycon 9 and glycoside 10 are tested. It is interesting that different from those from A. asphodeloides, the aglycon is an isospirostanol. It means that both isospirostanol and spirostanol could be antiviral if they have sugar chain at C3. Further examination of more isospirostanols 11-14 reveals an important SAR point that even small change on the aglycon skeleton like removal of H-5β could completely destroy the activities of isospirostanol glycosides.

### INDUSTRIAL APPLICABILITY

The present invention discloses a type of novel steroidal saponins and a method to prepare such steroidal saponins by extraction of Anemarrhena asphodeloides, and their anti-EV activity.

If desired, the different functions discussed herein may be performed in a different order and/or concurrently with each other. Furthermore, if desired, one or more of the above-described functions may be optional or may be combined.

While the foregoing invention has been described with respect to various embodiments and examples, it is understood that other embodiments are within the scope of the present invention as expressed in the following claims and their equivalents. Moreover, the above specific examples are to be construed as merely illustrative, and not limitative of the reminder of the disclosure in any way whatsoever. Without further elaboration, it is believed that one skilled in the art can, based on the description herein, utilize the present invention to its fullest extend. All publications recited herein are hereby incorporated by reference in their entirety.

What is claimed is:

1. A composition with anti-enteroviral effect comprising steroidal saponins extracted from Anemarrhena asphodeloides.

2. The composition according to claim 1 wherein the steroidal saponins comprising one or more of the following compounds: mangiferin, timosaponin B-II, anemarsaponin B, anemarsaponin II, timosaponin G, timosaponin A-IV and timosaponin A-III.

3. The composition according to claim 1 wherein the anti-enteroviral effects comprising effects of anti-enterovirus 71.

4. The composition according to claim 1 wherein the steroidal saponins is extracted and characterized by bioassay-guided counter-current chromatography.

5. The composition according to claim 4 wherein the counter-current chromatography is performed more than once.

6. The composition according to claim 4 wherein the bioassay-guided counter-current chromatography comprising continued solvent partition.

7. The composition according to claim 6 wherein the continued solvent partition comprising at least one two-phase solvent system incorporating ethyl acetate/butanol/water.

8. A method for treating enterovirus induced diseases comprising administering the composition according to claim 1.

9. The method according to claim 8 wherein the enterovirus induced diseases comprise diseases caused by enterovirus 71, and hand, foot and mouth disease.

10. A composition with anti-enteroviral effect comprising at least one of the following compounds: mangiferin, timosaponin B-II, anemarsaponin B, anemarsaponin II, timosaponin G, timosaponin A-IV and timosaponin A-III.
